(19) United States
(12) Patent Application Publication
(10) Pub. No.: US 2005/0117117 A1
(43) Pub. Date: Jun. 2, 2005

(54) Intraoperative biometry

(76) Inventor: Dan Bourla, Beit Arie (IL)

Correspondence Address: DEKEL PATENT LTD., DAVID KLEIN, BEIT HAROF'IM, 18 MENUHA VENAHALA STREET, ROOM 27, REHOVOT, 76209, IL

(21) Appl. No.: 10/724,821

(22) Filed: Dec. 2, 2003

(51) Int. Cl. A61B 18/18; A61B 3/10; G01B 11/02

(52) U.S. Cl. 351/221; 356/496; 606/10

(57) ABSTRACT

A biometer including a partial coherence interferometry (PCI) device connected to a microscope, the microscope being adapted to focus radiation from the PCI device to an eye.

### FIELD OF THE INVENTION

The present invention relates generally to biometry, such as optical biometry, and particularly to measuring features of an eyeball during surgery thereof and thereafter.

### BACKGROUND OF THE INVENTION

Intraocular lenses are used to replace a lens in a patient's eye in the case of cataracts. In such instances, it is standard practice before surgery to perform optical biometry, that is, the measurement of the optical axis length and refractive error (and possibly other optical features, such as but not limited to, the corneal curvature and anterior chamber depth (ACD)) of the patient's eye in order to provide information for calculation of an appropriate replacement for the lens. In many of these cases, however, it is not possible to use standard optical refractometry because the fundus is not visible through the turbid cataract eye lens. Standard optical refractometry suffers from the additional drawback that it does not measure the length of the patient's eye.

Ultrasound or acoustic biometry (also referred to as applanation acoustic biometry) is a method that is used to measure the length of the patient's eye. In performing ultrasound biometry, an ultrasound transducer is placed on the cornea of the patient's eye. Next, an ultrasound pulse is emitted by the transducer and is reflected back from the fundus of the eye. As is well known, the time of flight of the pulse depends on the length of, and the index of refraction of the ultrasound pulse in, the patient's eye. Using this information, the length of the patient's eye and the ACD can be determined. However, there is a drawback in using ultrasound biometry. In particular, in order to receive an echo that is strong enough to be measured with a suitable signal-to-noise ratio, the ultrasound transducer has to be brought into contact with the patient's eye and a special contact jelly may have to be used in certain cases. In addition, the ultrasound biometry requires the cooperation of the patient for good results. Also, operator dependent factors influence the measurement. This causes inaccuracies in the optical axis length and ACD measurement.

Another technique that is used is partial coherence interferometry. A device that uses this technology is commercially available from Carl Zeiss and is marketed under the name IOLMaster. The principles of partial coherence interferometry are discussed in such documents as U.S. Pat. No. 5,975,699 to Hellmuth, which describes a method and apparatus for simultaneously measuring the length and refractive error of an eye in a non-contact mode. The apparatus measures the length and refractive error of an eye which includes: (a) a source of short coherence radiation which couples radiation into a Michelson interferometer, the arms of the Michelson interferometer having a predetermined optical path length difference; (b) an injector which couples radiation output from the interferometer into the eye; and (c) a relay system which couples radiation output from the eye to a spectrometer; wherein the spectrometer measures displacement of radiation to measure the refractive error and the spectrometer measures density of fringes to measure the length.

Briefly, the term coherence describes the physical property of two wave fronts having a temporally constant or regularly varying phase difference at every point in space. The laser in the Michelson interferometer emits light (e.g., infrared light with a wavelength of 780 nm) of short coherence length (e.g., 160 microns), which is split into two partial beams of different optical path length. Both partial beams are reflected at the cornea and retina.

Interference occurs if the path difference between the partial beams is smaller than the coherence length. One leg contains the photodetector while the eye to be measured is arranged in the other leg of the interferometer. The interference signal received by the photodetector is measured with respect to the position of the interferometer mirror. The measured parameter is the optical path length between the cornea and the retina.

The device that works with the principle of partial coherence interferometry (PCI) is very accurate. However, it has the drawback of not being applicable for patients with different problems, such as but not limited to, significant axial opacity, dense posterior-subcapsular and mature cataracts, vitreous hemorrhage, and central corneal scar.

### SUMMARY OF THE INVENTION

The present invention seeks to provide an improved partial coherence interferometry (PCI) device that overcomes the problems of the prior art. As mentioned above, in the prior art measurements are taken prior to surgery, before removing the patient's lens, e.g., with phacoemulsification or other lens removal methods, or before removing a vitreous opacity, or replacing scarred corneal tissue with a clear corneal graft. In contrast, in the present invention, the PCI device is used during surgery after removal of the lens, or vitreal opacities, or scarred corneal tissue (and possibly thereafter), thereby providing a clear optical path to perform PCI, as is described more in detail hereinbelow.

It is noted that the invention is described herein with respect to light radiation. However, it is understood that the invention is not limited to this range of electromagnetic wave energy and may include other wavelengths of electromagnetic wave energy.

### BRIEF DESCRIPTION OF THE DRAWINGS

The present invention will be understood and appreciated more fully from the following detailed description taken in conjunction with the appended drawings in which:

FIG. 1 is a simplified schematic illustration of an optical biometer, constructed and operative in accordance with an embodiment of the present invention; and

FIG. 2 is a simplified flowchart of a method for biometry, such as optical biometry, in accordance with an embodiment of the present invention.

### DETAILED DESCRIPTION OF THE PRESENT INVENTION

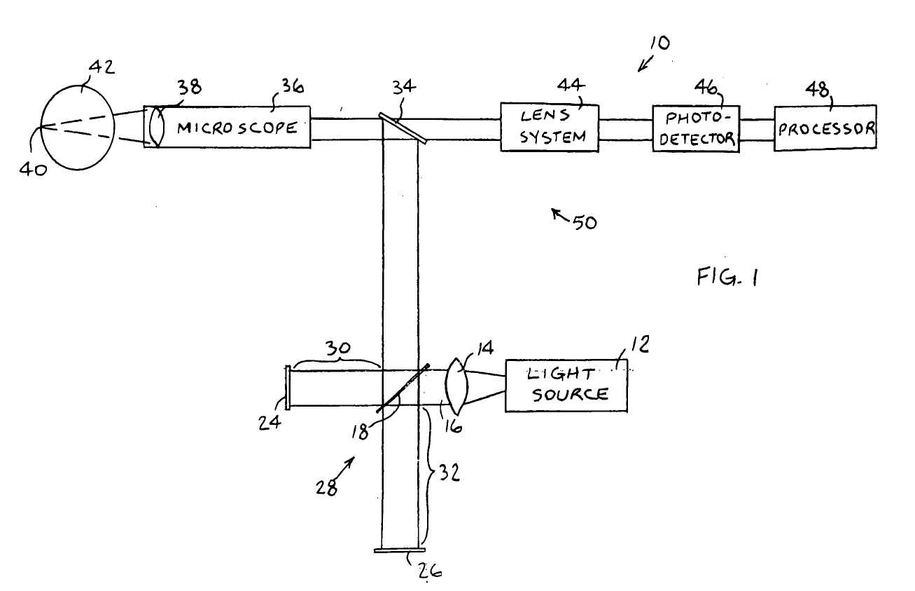

Reference is now made to FIG. 1, which illustrates an optical biometer 10, constructed and operative in accordance with an embodiment of the present invention.

Optical biometer 10 may comprise a short coherence light source 12, such as but not limited to, a superluminescent diode (SLD). Radiation output from light source 12 may be collimated by a lens system 14 into a collimated beam 16. Collimated beam 16 may impinge upon a beam splitter 18 and may be split into beams 20 and 22.

Beams 20 and 22 may impinge upon retroreflectors 24 and 26, which may be mirrors, for example. As is well understood by those of ordinary skill in the art, light source 12, lens system 14, beam splitter 18, and retroreflectors 24 and 26 are familiarly known in the art as a Michelson interferometer (“Michelson interferometer 28”). As described in U.S. Pat. No. 5,975,699 to Hellmuth, the difference between the optical path length of radiation traversing arms 30 and 32 of Michelson interferometer 28 may be chosen to equal the product of the length and refractive index of a reference or standard human eye. The index of refraction and length of a typical human eye is well known to those of ordinary skill in the art. For example, the index of refraction may be taken as 1.336 and the length may be taken as 24 mm.

The reflected beams may be combined by beam splitter 18 and impinge upon another beam splitter 34. Beam splitter 34 may direct a portion of the incident radiation towards a lens 36 of a microscope 38, such as but not limited to, any microscope suitable for use in cataract surgery. Microscope 38 may be used to focus the incident radiation onto the fundus 40 of an eye 42, thereby generating a secondary radiation (light) source on the fundus 40. The eye 42 is preferably an aphakic eye, that is, an eye from which the natural lens has been removed.

Radiation emanating from this secondary light source on fundus 40 may be collimated by the lens or lens system of microscope 38 (or lens system auxiliary to microscope 38 and used in conjunction therewith), wherein the collimated beam passes through beam splitter 34 and impinges upon a lens system 44, which may include various optical components, such as but not limited to, a relay lens system, a grating, and a diaphragm with decentered apertures axially displaced from the optical axis of the relay lens system, and focusing lens systems, for example. Light exiting lens system 44 may be detected by one or more photodetectors 46, such as but not limited to, CCD linear detectors. Output from photodetectors 46 may be input to a processor 48 (e.g., computer).

In short, the optical biometer 10 may comprise a partial coherence interferometry (PCI) device 50 connected to the microscope 38, wherein the PCI device 50 may include, for example, Michelson interferometer 28, beam splitter 34, lens system 44, one or more photodetectors 46 and processor 48.

The PCI device 50 may be coupled directly to microscope 38. Alternatively, PCI device 50 may be connected (e.g., mechanically, electromechanically and/or optically) to microscope 38, such that microscope 38 may be selectively moved into the line of sight of the eye or moved to the side out of the line of sight of the eye to allow using the PCI device 50 directly with the eye without the microscope 38.

The usage of the optical biometer 10 of the present invention is readily known and understood by persons skilled in the art, and is basically similar to the usage of the IOLMaster. Such usage is described in the user manual of the IOLMaster, readily available to the public, and may be understood also from U.S. Pat. No. 5,975,699 to Hellmuth. However, in the present invention, as opposed to the prior art, the optical biometer 10 comprises microscope 38 and may be used to measure an aphakic eye during or after surgery. The optical biometer 10 is thus not hindered by any of the prior art problems, such as but not limited to, significant axial opacity, dense posterior-subcapsular and mature cataracts, vitreous hemorrhage, and central corneal scar.

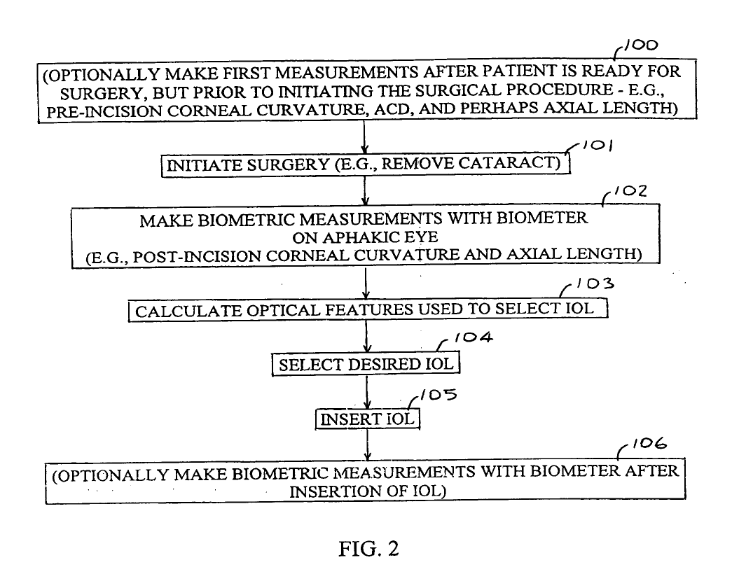

For example, as shown in FIG. 2, after a surgeon performs corneal incision and removes the cataract (reference numeral 101 in FIG. 2), the optical biometer 10 may be aimed and focused on the eye to make the necessary biometric measurements (reference numeral 102). The processor 48 is adapted to calculate the optical features used in the selection of an intraocular lens (IOL), such as but not limited to, the optical axis length, refractive error and corneal curvature (e.g., on the post-incision cornea) (reference numeral 103). A desired IOL may be selected based on these measurements (reference numeral 104) and then inserted in the eye (reference numeral 105). Since the measurements may be performed at the aphakic stage of the operation, the measurements are not be affected by dense lens opacities. The PCI device may also be used to make biometric measurements after insertion of an IOL into the eye (reference numeral 106). The PCI device may also be used in the pre-corneal incision stage of the operation in order to obtain the ACD and pre-incision corneal curvature, which may also be used in the calculation and selection of the IOL.

The ACD is a factor is a measurement of the distance between the corneal surface and the anterior surface of the lens. Certain theoretical formulas for calculating IOL power utilize this measurement for estimating the Effective Lens Position (ELP) of the implanted IOL. Since the final position of the IOL has a direct effect on the required IOL power, this measurement is useful for eyes with very deep or very shallow ACDs.

Although the PCI device 50 may be used to measure the optical axial length at the aphakic stage of the operation, the ACD cannot readily be obtained, as there is no anterior lens surface to reference the measurement. Consequently, the following procedure may be used: The first measurement may be performed after the patient is ready for surgery, but prior to initiating the surgical procedure (intraoperative, pre-incisional measurement). This measurement may provide the pre-incision corneal curvature, ACD, and in a few cases the desired axial length (reference numeral 100). A second measurement may be made at the clear media (aphakic) stage of the operation (reference numeral 101), providing the post-incision corneal curvature and axial length (intraoperative, post-incisional measurement).

This procedure may not only enable obtaining the ACD, but may also provide a factor of the corneal curvature's drift from the pre- to post-incision stage, and thus allow for a more precise mathematical calculation of the estimated final postoperative corneal curvature. It is believed that neither the pre-incision nor the post-incision corneal curvature fully reflects the final corneal curvature. As such, the method of the present invention may provide additional information, which may be used by future calculation methods to arrive at the final corneal curvature.

Although the invention has been described in conjunction with specific embodiments thereof, many alternatives, modifications and variations fall within the spirit and broad scope of the appended claims.

1. A biometer comprising:
a partial coherence interferometry (PCI) device connected to a microscope, said microscope being adapted to focus radiation from the PCI device to an eye.

2. The biometer according to claim 1, wherein said PCI device comprises an interferometer that directs a beam to a beam splitter, said beam splitter directing a portion of radiation incident thereon towards a lens of said microscope.

3. The biometer according to claim 2, further comprising a lens system, wherein said microscope is adapted to focus radiation incident thereon to a portion of an eye to generate a secondary radiation source on the portion of the eye, wherein radiation emanating from the secondary radiation source passes through said beam splitter and impinges upon said lens system.

4. The biometer according to claim 3, further comprising at least one photodetector adapted to detect radiation exiting said lens system.

5. The biometer according to claim 4, further comprising a processor in communication with and adapted to process an output from said at least one photodetector.

6. The biometer according to claim 2, wherein said interferometer comprises a Michelson interferometer, and a difference between path lengths of radiation traversing arms of the Michelson interferometer equals the product of the length and refractive index of a reference eye.

7. A method for performing biometry, comprising:
removing a lens from an eye; and
making biometric measurements with a PCI device aimed at the eye after removal of the lens therefrom.

8. The method according to claim 7, further comprising calculating optical features based on the biometric measurements and selecting an intraocular lens (IOL) in accordance with the optical features.

9. The method according to claim 7, further comprising making biometric measurements with the PCI device aimed at the eye after insertion of an IOL into the eye.

10. The method according to claim 7, further comprising making pre-incision biometric measurements with the PCI device.
